(19) United States
(12) Patent Application Publication
(10) Pub. No.: US 2015/0342626 A1
(43) Pub. Date: Dec. 3, 2015

(54) CALCULUS AIMING AND LOCKING SYSTEM

(71) Applicant: Lite-Med Inc., Taipei (TW)

(72) Inventor: Chen-Ming HSU, Taipei (TW)

(73) Assignee: Lite-Med Inc.

(21) Appl. No.: 14/337,906

(22) Filed: Jul. 22, 2014

(30) Foreign Application Priority Data
May 27, 2014 (TW) TW103118464

(51) Int. Cl. A61B 17/22

(52) U.S. Cl. CPC A61B 17/22004

(57) ABSTRACT

The invention is a calculus aiming and locking system that is an application computer system and comprises a calculus locating module, an aiming and locking and emission control module, and a shockwave generation module. The calculus locating module acquires and calculates a calculus image of a calculus and the aiming and locking and emission control module determines whether to trigger a shockwave generation module to control a shockwave emission device to emit energy at an effective aiming area according to whether the calculus coordinate is within the effective aiming area. The invention renders the calculus hit rate 100% when the shockwave emission device emits energy, which may save energy substantially and avoid causing injuries to the normal tissue of the patient requiring extracorporeal shockwave lithotripsy treatment.

### BACKGROUND OF THE INVENTION

1. Technical Field

The invention relates to a calculus aiming and locking system, and more particularly, to a calculus aiming and locking system that may lock calculi so that the extracorporeal shockwave lithotripter hit rate may reach 100%.

2. Description of Related Art

Present extracorporeal shockwave lithotripsy medical devices utilize a shockwave lithotripter to continuously emit energy of shockwaves with a fixed frequency for a period of time at a patient on a treatment table.

Although a X-ray imaging system or an ultrasound imaging system is used to acquire the image of the calculus and let the shockwave lithotripter aim at the location of the calculus before the shockwave lithotripsy treatments, since the human body may breathe and move, the movements due to breathing or feeling unwell during the shockwave lithotripsy treatment may cause the energy emitted from the shockwave lithotripter to be directed at normal tissue. Such may cause illness or inflammation while even more severe cases may cause bleeding or rupture of the tissue, which not only has side effects but also may result in serious medical malpractice claims.

In view of this, the invented extracorporeal shockwave lithotripter and its calculus tracking system in TW Patent No. I279221 may sharply and effectively track and position the calculi in human bodies and contribute profoundly to medical industry.

However, if a calculus aiming and locking system that directs all the energy emitted by the shockwave lithotripter onto only the calculus by aiming and locking the calculus after locating may be devised and realized, it shall be looked forward to and largely welcomed by users of extracorporeal shockwave lithotripsy medical devices and medical technology and equipment industries.

### SUMMARY OF THE INVENTION

The present invention is a calculus aiming and locking system and is an application computer system, comprising: a calculus locating module, an aiming and locking and emission control module, and a shockwave generation module. The invention renders the calculus hit rate 100% when the shockwave emission device emits energy, which may save energy substantially and avoid causing unnecessary injuries that result from the shockwave emission device emitting energy directed to the normal tissue of the patient.

The invention provides a calculus aiming and locking system, wherein the calculus aiming and locking system is an application computer system, comprising: a calculus locating module, acquiring a calculus image of a calculus and using a characteristic point of the calculus image to calculate a calculus coordinate corresponding to the characteristic point; and an aiming and locking and emission control module, inputted with the calculus coordinate from the calculus locating module, comparing the calculus coordinate with an effective aiming area and controlling a shockwave generation module to trigger a shockwave emission device to emit energy at the effective aiming area when the calculus coordinate is within the effective aiming area.

Implementation of the present invention at least involves the following inventive steps:

(1) the hit rate of the shockwave emission device may reach 100%;

(2) massive energy may be saved; and

(3) injuries of the normal tissue of the patient during shockwave lithotripsy treatments are largely reduced.

The features and advantages of the present invention are detailed hereinafter with reference to the preferred embodiments. The detailed description is intended to enable a person skilled in the art to gain insight into the technical contents disclosed herein and implement the present invention accordingly. In particular, a person skilled in the art can easily understand the objects and advantages of the present invention by referring to the disclosure of the specification, the claims, and the accompanying drawings.

### BRIEF DESCRIPTION OF THE DRAWINGS

The invention as well as a preferred mode of use, further objectives and advantages thereof will be best understood by reference to the following detailed description of illustrative embodiments when read in conjunction with the accompanying drawings, wherein:

FIG. 1 is a schematic view of an example of the system block diagram of a calculus aiming and locking system of the present invention;

FIG. 2 is a schematic view of an example of a calculus coordinate and an effective aiming area of the present invention;

FIG. 3 is a schematic view of an example of the process steps performed by the aiming and locking and emission control module of the present invention;

FIG. 4 is another schematic view of an example of the system block diagram of a calculus aiming and locking system of the present invention; and

FIG. 5 is a schematic view of an example of the process steps performed by the calculus locating module of the present invention;

### DETAILED DESCRIPTION OF THE INVENTION

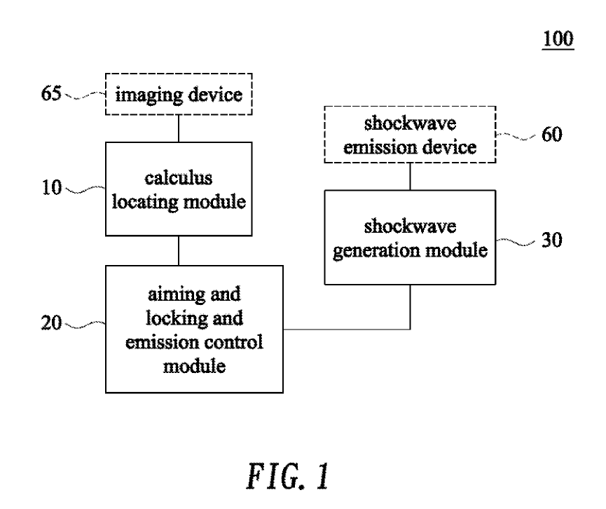

FIG. 1 shows an embodiment of the calculus aiming and locking system 100, comprising a calculus locating module 10, an aiming and locking and emission control module 20, and a shockwave generation module 30. The calculus aiming and locking system 100 is an application computer system and may be a personal computer (PC), industrial PC (IPC), embedded PC (EPC), single-board PC, etc., and may be a combination of software, hardware, and application programs. Advantages may include easy assembly, flexible applications, low construction cost, and small required space and is suitable to be combined with shockwave lithotripsy medical devices for usage.

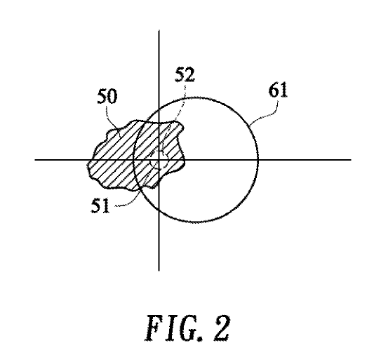

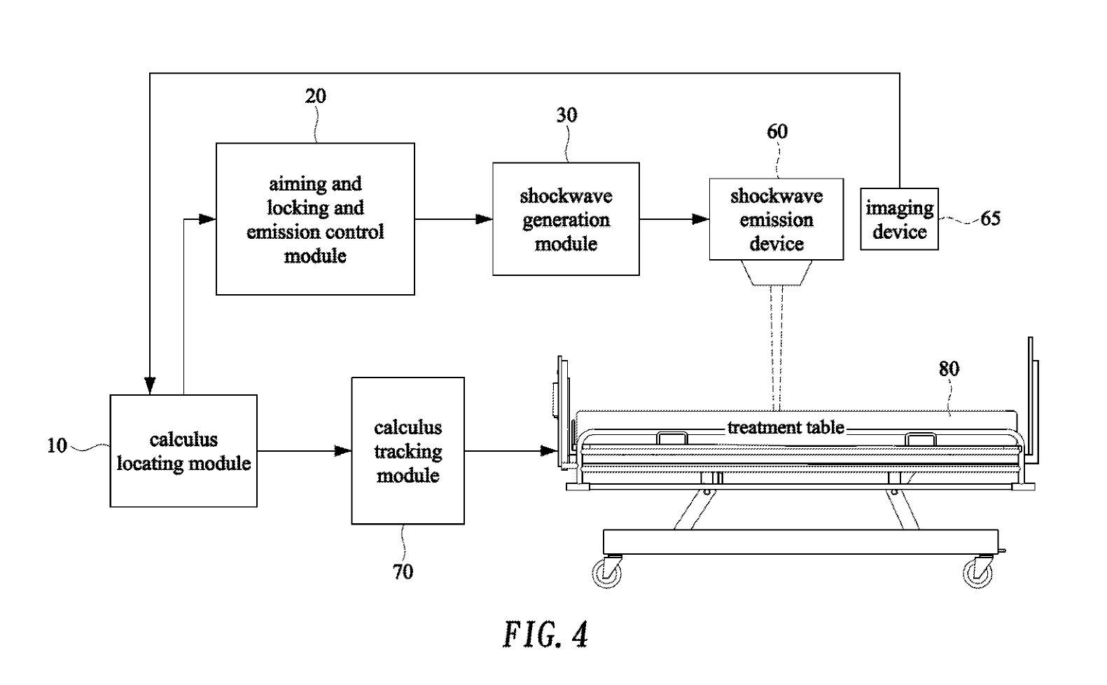

As shown in FIG. 1, FIG. 2, and FIG. 4, the calculus locating module 10 may acquire a calculus image 50 of a calculus from an external imaging device 65 and use a characteristic point 52 of the calculus image 50 to calculate a calculus coordinate 51 corresponding to the characteristic point 52, wherein the characteristic point 52 may be a brightest point of the calculus image 50 or a relative bright point of the calculus image 50 designated by a user.

After the calculus locating module 10 acquires the calculus image 50, the characteristic point 52 of the calculus image 50 is determined by software calculation, hardware comparison, or the combination of the two. Subsequently, the calculus coordinate 51 corresponding to the characteristic point 52 is calculated and outputted.

As also shown in FIG. 1 and FIG. 2, the aiming and locking and emission control module 20 is inputted with the calculus coordinate 51 from the calculus locating module 10 and compares the calculus coordinate 51 with an effective aiming area 61 set by the calculus aiming and locking system 100 and determines whether the calculus coordinate 51 is within the effective aiming area 61.

The effective aiming area 61 may be a predetermined area or a focus area of the shockwave emission device 60 combined with the calculus aiming and locking system 100 for usage. The effective aiming area 61 may be a circular area with a diameter between 11 mm and 13 mm, although it may be changed according to actual requirements of application or different shockwave emission devices 60 combined with the calculus aiming and locking system 100 for usage.

As also shown in FIG. 1 and FIG. 2, the shockwave generation module 30 is connected to the aiming and locking and emission control module 20 and is triggered by the aiming and locking and emission control module 20 to control the shockwave emission device 60 to emit energy at the effective aiming area 61.

The shockwave emission device 60 emits energy at the effective aiming area 61 by triggering the shockwave emission device 60 via the shockwave generation module 30 to emit energy at the effective aiming area 61 with a specified frequency when the aiming and locking and emission control module 20 successfully aims at the calculus continuously, wherein the specified frequency may be designated to be between 60 times per minute and 240 times per minute.

Besides, the aiming and locking and emission control module 20 determines whether the calculus has been aimed at and locked according to whether the calculus coordinate 51 is within the effective aiming area 61, and emits shockwave at the calculus or enters standby and continues to carry out comparison and determination regarding the calculus.

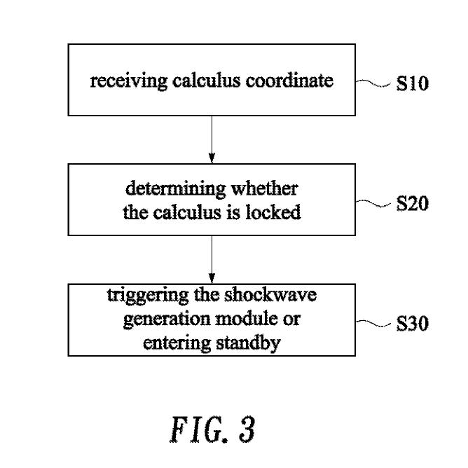

In other words, as shown in FIG. 3, the aiming and locking and emission control module 20 may repeat the steps of: receiving calculus coordinate (step S10); determining whether the calculus is locked (step S20); and triggering the shockwave generation module or entering standby (step S30).

Receiving calculus coordinate (step S10) is the transmission of the calculus coordinate 51 from the calculus locating module 10 to the aiming and locking and emission control module 20 as described in above.

Determining whether the calculus is locked (step S20) is for the aiming and locking and emission control module 20 to determine whether the calculus coordinate 51 is within the effective aiming area 61.

Triggering the shockwave generation module or entering standby (step S30) depends on the results of the determination of whether the calculus is locked (step S20); the aiming and locking and emission control module 20 triggers the shockwave generation module 30 to emit energy at the calculus when the calculus is determined to be locked (“yes” in step S20), or enters standby for a specified time when the calculus is determined not to be locked (“no” in step S20), wherein the specified time of entering standby may be set to be between 1 ms (millisecond) and 1000 ms according to requirements of application.

In addition, as shown in FIG. 4, the calculus aiming and locking system 100 may further comprise a calculus tracking module 70, which is connected with the calculus locating module 10 and receives input of the calculus coordinate 51 from the calculus locating module 10.

The calculus tracking module 70 as shown in FIG. 4 may calculate an offset and an offset direction or angle of the calculus coordinate 51 with respect to a center of the effective aiming area 61 and control the movement of a treatment table 80 connected with the calculus aiming and locking system 100 according to the calculated offset and offset direction or angle.

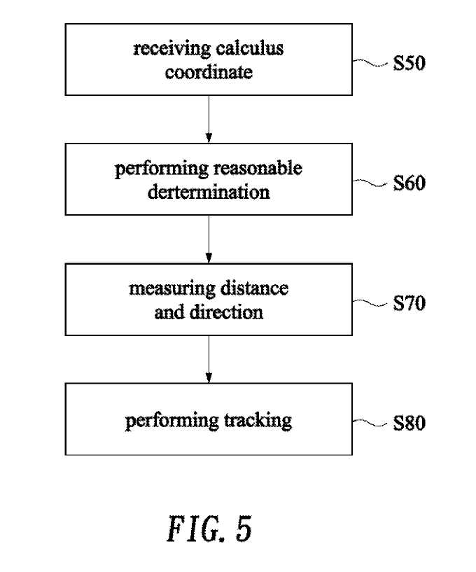

As shown in FIG. 5, the calculus tracking module 70 may repeat the steps of: receiving calculus coordinate (step S50); performing reasonable determination (step S60); measuring distance and direction (step S70); and performing tracking (step S80).

As shown in FIG. 4 and FIG. 5, receiving calculus coordinate (step S50) is for the calculus tracking module 70 to input the calculus coordinate 51 from the calculus locating module 10.

As also shown in FIG. 4 and FIG. 5, performing reasonable determination (step S60) is the confirmation by the calculus tracking module 70 that the distance of the calculus coordinate 51 from the center of the effective aiming area 61 is less than 100 pixels, where the pixels here refer to the pixels of the display connected to the calculus aiming and locking system 100.

Performing reasonable determination (step S60) is necessary since when the distance between the calculus coordinate 51 and the center of the effective aiming area 61 is too large, the moving range of the treatment table 80 controllable by the calculus tracking module 70 may be exceeded, and the location of the treatment table 80 must be readjusted and the calculus aiming and locking system 100 must be restarted,

As also shown in FIG. 4 and FIG. 5, measuring distance and direction (step S70) is to measure the distance and direction of the calculus coordinate 51 from the center of the effective aiming area 61 by the calculus tracking module 70 and to calculate the offset and the offset direction with respect to the center of the effective aiming area 61.

As also shown in FIG. 4 and FIG. 5, performing tracking (step S80) is to control the movement of the treatment table 80 according to the offset and the offset direction calculated by the calculus tracking module 70 so that the distance of the calculus coordinate 51 from the center of the effective aiming area 61 is less than 1 mm to track the calculus effectively.

In summary, when the calculus coordinate 51 representing the location of the calculus is not at the center of the effective aiming area 61, the calculus tracking module 70 controls the movement of the treatment table 80 so that the calculus coordinate 51 representing the location of the calculus may be as close as possible to the center of the effective aiming area 61 (moved so that the distance from the center of the effective aiming area 61 is less than 1 mm). Therefore, in addition to aiming and locking calculi, the calculus aiming and locking system 100 further has functions to track the calculi which renders the calculus aiming and locking system 100 more efficient in shockwave lithotripsy treatments.

The embodiments described above are intended only to demonstrate the technical concept and features of the present invention so as to enable a person skilled in the art to understand and implement the contents disclosed herein. It is understood that the disclosed embodiments are not to limit the scope of the present invention. Therefore, all equivalent changes or modifications based on the concept of the present invention should be encompassed by the appended claims.

What is claimed is:

1. A calculus aiming and locking system, wherein the calculus aiming and locking system is an application computer system, comprising:
a calculus locating module, acquiring a calculus image of a calculus and using a characteristic point of the calculus image to calculate a calculus coordinate corresponding to the characteristic point; and
an aiming and locking and emission control module, inputted with the calculus coordinate from the calculus locating module, comparing the calculus coordinate with an effective aiming area and controlling a shockwave generation module to trigger a shockwave emission device to emit energy at the effective aiming area when the calculus coordinate is within the effective aiming area.

2. The calculus aiming and locking system as claimed in claim 1, wherein the characteristic point is a brightest point of the calculus image or a relative bright point designated by a user.

3. The calculus aiming and locking system as claimed in claim 1, wherein the effective aiming area is a circular area with a diameter between 11 mm and 13 mm.

4. The calculus aiming and locking system as claimed in claim 1, wherein the shockwave generation module controls the shockwave emission device to emit energy at the effective aiming area with a specified frequency.

5. The calculus aiming and locking system as claimed in claim 4, wherein the specified frequency is between 60 times per minute and 240 times per minute.

6. The calculus aiming and locking system as claimed in claim 1, wherein the aiming and locking and emission control module repeats the steps of:
receiving calculus coordinate;
determining whether the calculus is locked, which is performed by the aiming and locking and emission control module to determine whether the calculus coordinate is within the effective aiming area; and
triggering the shockwave generation module or entering standby, wherein the aiming and locking and emission control module triggers the shockwave generation module when the calculus is determined to be locked, or enters standby for a specified time when the calculus is determined to be not locked.

7. The calculus aiming and locking system as claimed in claim 6, wherein the specified time is between 1 ms (millisecond) and 1000 ms.

8. The calculus aiming and locking system as claimed in claim 1, further comprising a calculus tracking module connected with the calculus locating module, wherein the calculus tracking module calculates an offset and an offset direction of the calculus coordinate with respect to a center of the effective aiming area and moves a treatment table connected with the calculus aiming and locking system according to the offset and the offset direction.

9. The calculus aiming and locking system as claimed in claim 8, wherein the calculus tracking module repeats the steps of:
receiving calculus coordinate, which is for the calculus tracking module to input the calculus coordinate from the calculus locating module;
performing reasonable determination, which is for the calculus tracking module to confirm that the distance of the calculus coordinate from the center of the effective aiming area is less than 100 pixels;
measuring distance and direction, which is to measure the distance and direction of the calculus coordinate from the center of the effective aiming area and calculating the offset and the offset direction with respect to the center of the effective aiming area; and
performing tracking, which is to track the calculus by controlling the movement of the treatment table according to the offset and the offset direction, so that the distance of the calculus coordinate from the center of the effective aiming area is less than 1 mm.
